(19) United States
(12) Patent Application Publication
(10) Pub. No.: US 2019/0203303 A1
(43) Pub. Date: Jul. 4, 2019

(54) CLASSIFICATION OF SUBTYPES OF KIDNEY TUMORS USING DNA METHYLATION

(71) Applicants: Sameer Chopra, Los Angeles, CA (US); Jie Liu, San Mateo, CA (US); Inderbir Singh Gill, Pasadena, CA (US); Kimberly Siegmund, San Marino, CA (US)

(72) Inventors: Sameer Chopra, Los Angeles, CA (US); Jie Liu, San Mateo, CA (US); Inderbir Singh Gill, Pasadena, CA (US); Kimberly Siegmund, San Marino, CA (US)

(21) Appl. No.: 16/314,335

(22) PCT Filed: Jun. 28, 2017
(86) PCT No.: PCT/US2017/039795

§ 371 Date: Dec. 28, 2018

Related U.S. Application Data
(60) Provisional application No. 62/356,204, filed on Jun. 29, 2016.

(51) Int. Cl. C12Q 1/6886; C12Q 1/6827; C12Q 1/6858; G16H 50/50; G16H 50/20

(52) U.S. Cl. CPC C12Q 1/6886; C12Q 1/6827; C12Q 1/6858; C12Q 2600/156; G16H 50/20; C12Q 2600/112; C12Q 2600/154; G16H 50/50

(57) ABSTRACT

A method of classifying kidney tumors is provided. The method includes obtaining a sample from a subject, isolating DNA from the sample, determining the methylation status of the DNA, and comparing the methylation status of the DNA to one or more GO methylated biomarkers selected from the following: cg04877910, cg09667289, cg05274650, cg1 1473616, cg16935734, cg27534624, cg21851713, cg15867829, cg15679829, cg08884979, cg09538401, cg26811868, cg05367028, cg19816080, cg20108357, cg25504868, cg1 1201447, cg19922137, cg14706317, cg15902830, cg10794973, cg10777887, cg03290131, cg07851269, cg1 1264947, cg00279406, cg23140965, cg03574652, cg03265671, cg24864241, cg01572891, cg00193963, cg14329285, cg17819990, cg17298239, cg23856138, cg21049501, cg1 1808936, cg25170591, cg17983632, cg08141142, cg19848599, cg25799109, cg07093324, cg16223546, cg07604732, cg12149606, cg08949329, cg27166177, cg26177041, cg09885851, cg22876153, cg21386992, cg02309772, cg02833180, cg20007890, cg04972244, cg02666955 and cg12102682. The comparison indicates whether the sample is clear cell malignant, papillary malignant, chromophobe malignant, angiomylolipomas (AML) benign, or oncocytoma benign.

### RELATED APPLICATIONS

This application claims the benefit of U.S. Provisional Application No. 62/356,204, filed Jun. 29, 2016, the entire contents of which are incorporated herein by reference in its entirety.

### STATEMENT REGARDING FEDERALLY SPONSORED RESEARCH OR DEVELOPMENT

This invention was made with government support under National Institutes of Health grant R21 CA167367. The government has certain rights in the invention.

### FIELD OF THE INVENTION

The present invention relates to methods of screening and classifying kidney tumors.

### BACKGROUND OF THE INVENTION

It is estimated that 62,700 new cases of renal cancer will be diagnosed in 2016 [1]. The incidence in the US has increased significantly over the past 10 years [2] due to increased use of abdominal imaging. However, although the incidence of renal cell carcinoma (RCC) is increasing, the mortality from this disease has not increased proportionately [1]. This is attributed both to the increased detection of localized small renal masses (SRMs), which are classified as tumors measuring <4 cm in diameter and account for 48-66% of new kidney cancers [3]. In addition, 30% of SRMs are benign [4] and many SRMs having a low malignant potential. This is concerning as it has led to over diagnosis and over treatment for indolent lesions [5]. Nearly 65% of all renal masses are diagnosed when they are localized, and it has been shown that the incidence of benign pathology is inversely related to tumor size (i.e., a decrease in renal mass size increases the frequency of benign pathology) [6]. Current imaging techniques alone are unable to definitively distinguish benign from malignant pathologies [7]. Despite this, the majority of SRMs are still being treated without a pretreatment diagnostic biopsy, causing significant unnecessary morbidity to patients. Thus, renal tumor biopsies have the potential to assist in both the histological assessment and management of patients [3].

While radiologic imaging provides clues as to the pathology of the mass, incidental non-neoplastic findings such as trauma, infection, hemorrhage, infections, and cysts have radiographic features that occasionally are from those of the spectrum of renal carcinomas [7]. Furthermore, malignant and benign lesions appear to grow at similar rates, therefore this parameter cannot accurately identify malignant lesions requiring early intervention [8]. Currently, needle biopsies have been used along with radiologic assessment to evaluate SRMs, however, the applicability and the diagnostic and predictive accuracy of needle biopsy remain in question [9-11]. The accuracy of needle biopsy in distinguishing benign from malignant lesions ranges from 73-94%, but in SRMs, the needle biopsies have lower specificity, sensitivity, and a high rate of false negativity [11].

It has been postulated that combining histological results with molecular markers can improve the sensitivity of needle biopsies. While mRNA and protein-based markers are promising, in the SRM clinical scenario, the small amount of tissue available from the needle biopsy, sample stability issues, and the associated costs for subsequent analysis present significant challenges that make these markers burdensome choices.

DNA methylation alterations are among the first changes to occur in the process of tumorigenesis [12]. Because of this, it is likely that they will be present in the majority of tumors, as well as in less aggressive malignancies. Furthermore, they are easily detected in needle biopsy samples. DNA methylation is a stable modification from a stable DNA molecule, and therefore is less likely to be degraded in clinical samples. At the same time, PCR-based approaches allow for the analysis of DNA methylation using a very small sample with low costs. In fact, DNA methylation markers are currently being utilized to detect tumors in serum and urine sediments [13-16]. The fact that DNA methylation changes occur in RCC [17, 18] coupled with the ease of its detection, warrants further investigation to determine the applicability of utilizing DNA methylation markers to improve the accuracy of needle biopsies in SRMs in a clinical setting.

### SUMMARY OF THE INVENTION

One aspect of the present invention is directed to a method of classifying kidney tumors. The method includes obtaining a sample from a subject, isolating DNA from the sample, determining the methylation status of the DNA and comparing the methylation status of the DNA to one or more methylated biomarkers selected from the following: cg04877910, cg09667289, cg05274650, cg11473616, cg16935734, cg27534624, cg21851713, cg15867829, cg15679829, cg08884979, cg09538401, cg26811868, cg05367028, cg19816080, cg20108357, cg25504868, cg11201447, cg19922137, cg14706317, cg15902830, cg10794973, cg10777887, cg03290131, cg07851269, cg11264947, cg00279406, cg23140965, cg03574652, cg03265671, cg24864241, cg01572891, cg00193963, cg14329285, cg17819990, cg17298239, cg23856138, cg21049501, cg11808936, cg25170591, cg17983632, cg08141142, cg19848599, cg25799109, cg07093324, cg16223546, cg07604732, cg12149606, cg08949329, cg27166177, cg26177041, cg09885851, cg22876153, cg21386992, cg02309772, cg02833180, cg20007890, cg04972244, cg02666955 and cg12102682. The methylated biomarker includes a sequence region that extends up to 250 base pairs upstream and downstream from the methylated biomarker. The comparison indicates whether the sample is clear cell malignant, papillary malignant, chromophobe malignant, angiomylolipomas (AML) benign, or oncocytoma benign.

Examples of methylation sensitive assays that can be used to determine the DNA methylation status include but are not limited to HM450, HM850, real-time methylation sensitive PCR (MSP), MethyLight and Pyrosequencing.

In one embodiment, the sample is a biopsy sample including liquid biopsy (circulating tumor cells, CTC or circulating tumor DNA, ctDNA).

In another embodiment, the biopsy is from a small renal mass (SRM).

In another embodiment, two or more methylated biomarkers are selected.

In another embodiment, the sample is selected from the following: blood, plasma and urine.

In another embodiment, the sequence region extends up to 100 base pairs upstream and downstream from the methylated biomarker.

In another embodiment, the sequence region extends 0 base pairs upstream and downstream from the methylated biomarker.

In another embodiment, five or more methylated biomarkers are selected.

In another embodiment, fifteen or more methylated probes are selected.

Another aspect of the present invention is directed to a method of identifying subjects having renal cancer. The method includes obtaining a sample from a subject, isolating DNA from the sample, determining the methylation status of the DNA and comparing the methylation status of the DNA to one or more methylated biomarkers selected from the following: cg04877910, cg09667289, cg05274650, cg11473616, cg16935734, cg27534624, cg21851713, cg15867829, cg15679829, cg08884979, cg09538401, cg26811868, cg05367028, cg19816080, cg20108357, cg25504868, cg11201447, cg19922137, cg14706317, cg15902830, cg10794973, cg10777887, cg03290131, cg07851269, cg11264947, cg00279406, cg23140965, cg03574652, cg03265671, cg24864241, cg01572891, cg00193963, cg14329285, cg17819990, cg17298239, cg23856138, cg21049501, cg11808936, cg25170591, cg17983632, cg08141142, cg19848599, cg25799109, cg07093324, cg16223546, cg07604732, cg12149606, cg08949329, cg27166177, cg26177041, cg09885851, cg22876153, cg21386992, cg02309772, cg02833180, cg20007890, cg04972244, cg02666955 and cg12102682. The comparison indicates whether the sample is clear cell malignant, papillary malignant, chromophobe malignant, angiomylolipomas (AML) benign, or oncocytoma benign. The methylated biomarker includes a sequence region that extends up to 250 base pairs upstream and downstream from the methylated biomarker. The comparison indicates whether the sample is normal or malignant.

In one embodiment, the sample is a biopsy sample including liquid biopsy (CTC or ctDNA).

In another embodiment, the biopsy is from a small renal mass (SRM).

In another embodiment, two or more methylated biomarkers are selected.

In another embodiment, the sample is selected from the following: blood, plasma and urine.

In another embodiment, the sequence region extends up to 100 base pairs upstream and downstream from the methylated biomarker.

In another embodiment, the sequence region extends 0 base pairs upstream and downstream from the methylated biomarker.

In another embodiment, five or more methylated biomarkers are selected.

Another aspect of the present invention is directed to a composition comprising one or more methylated biomarkers selected from the following: cg04877910, cg09667289, cg05274650, cg11473616, cg16935734, cg27534624, cg21851713, cg15867829, cg15679829, cg08884979, cg09538401, cg26811868, cg05367028, cg19816080, cg20108357, cg25504868, cg11201447, cg19922137, cg14706317, cg15902830, cg10794973, cg10777887, cg03290131, cg07851269, cg11264947, cg00279406, cg23140965, cg03574652, cg03265671, cg24864241, cg01572891, cg00193963, cg14329285, cg17819990, cg17298239, cg23856138, cg21049501, cg11808936, cg25170591, cg17983632, cg08141142, cg19848599, cg25799109, cg07093324, cg16223546, cg07604732, cg12149606, cg08949329, cg27166177, cg26177041, cg09885851, cg22876153, cg21386992, cg02309772, cg02833180, cg20007890, cg04972244, cg02666955 and cg12102682.

In one embodiment, the composition is used in an assay to determine whether a sample is clear cell malignant, papillary malignant, chromophobe malignant, angiomylolipomas (AML) benign, or oncocytoma benign.

In another embodiment, the composition is used in an assay to determine whether a sample is normal or malignant.

Other aspects and advantages of the invention will be apparent from the following description and the appended claims.

### BRIEF DESCRIPTION OF THE DRAWINGS

FIG. 1. Multidimensional scaling plot of 697 training samples using the 500 features with greatest median absolute deviation.

FIG. 2. Training data set heatmap of 600 differentially methylated features (rows) in 697 kidney samples (columns). Columns are ordered by tissue subtype, and rows are ordered by sets of predictive features. Within each feature set, rows are ordered by average DNA methylation level in normal kidney.

FIG. 3. Six predicted probabilities for 272 ex vivo needle biopsy samples (102 normal kidney, 15 AML, 26 oncocytoma, 98 clear cell, 14 papillary, 6 chromophobe, 11 other benign). The probabilities are ordered by subgroup and the probability the sample is assigned to the correct subgroup.

FIG. 4. Fraction of 100 subtype-predictive features showing the attribute of interest. Reference is the 351124 features that remained after filtering.

FIG. 5. Six predicted probabilities for 697 kidney training samples (283 clear cell carcinomas, 81 papillary carcinomas, 65 chromophobe, 27 angiomylolipomas, 37 oncocytomas, and 204 normal kidney).

FIG. 6. Boxplots of the entropy for each sample (−Σi pi*ln(pi) where pi is the estimated probability of group i, i=1, . . . , 6). Top left is overall, Left middle is for samples with subtype incorrectly predicted, left bottom is for samples with subtype correctly predicted. Top Right is for samples with malignancy incorrectly predicted and Right middle is for samples with malignancy correctly predicted.

### DETAILED DESCRIPTION OF THE INVENTION

A “biomarker” as used herein refers to a molecular indicator that is associated with a particular pathological or physiological state. The “biomarker” as used herein is a molecular indicator for cancer, more specifically an indicator for renal cancer.

As used herein the term “cancer” refers to or describes the physiological condition in mammals that is typically characterized by abnormal and uncontrolled cell division or cell growth.

As used herein, a “subject” is preferably a human, non-human primate, cow, horse, pig, sheep, goat, dog, cat, or rodent. In all embodiments, human subjects are preferred. The “subject” may be at risk of developing kidney cancer or renal cell carcinoma (RCC), may be suspected of having kidney cancer or RCC, or may kidney cancer or RCC. In addition, a “subject” may simply be a person who wants to be screened for kidney cancer or RCC.

In this invention, available DNA methylation data from The Cancer Genome Atlas (TCGA) in subtypes of renal tumors is used and a classification model to predict subtypes of kidney tumor that include benign and malignant is built. Finally, we applied the classifier to predict both the malignancy and tissue subtype on 272 ex vivo biopsies from 100 RMs (73 renal masses were SRM). Overall, we demonstrate that cancer-specific DNA methylation data can be used as subtype-specific RCC biomarkers in needle biopsy specimens, which have potential utility in clinical decision-making, especially in SRMs. These markers could also be used in liquid biopsy of RCC.

One or more embodiments of the invention may use a computer. For instance, any of the DNA methylation status determinations and comparisons may be implemented, stored or processed by a computer. Further, any determination, evaluation or conclusion may likewise be derived, analyzed or reported by a computer. The type computer is not particularly limited regardless of the platform being used. For example, a computer system generally includes one or more processor(s), associated memory (e.g., random access memory (RAM), cache memory, flash memory, etc.), a storage device (e.g., a hard disk, an optical drive such as a compact disk drive or digital video disk (DVD) drive, a flash memory stick, magneto optical discs, solid state drives, etc.), and numerous other elements and functionalities typical of today's computers or any future computer. Each processor may be a central processing unit and may or may not be a multi-core processor. The computer may also include input means, such as a keyboard, a mouse, a tablet, touch screen, a microphone, a digital camera, a microscope, etc. Further, the computer may include output means, such as a monitor (e.g., a liquid crystal display (LCD), a plasma display, or cathode ray tube (CRT) monitor). The computer system may be connected to a network (e.g., a local area network (LAN), a wide area network (WAN) such as the Internet, or any other type of network) via a network interface connection, wired or wireless. Those skilled in the art will appreciate that many different types of computer systems exist, and the aforementioned input and output means may take other forms including handheld devices such as tablets, smartphone, slates, pads, PDAs, and others. Generally speaking, the computer system includes at least the minimal processing, input, and/or output means necessary to practice embodiments of the invention.

Further, those skilled in the art will appreciate that one or more elements of the aforementioned computer system may be located at a remote location and connected to the other elements over a network. Further, embodiments of the invention may be implemented on a distributed system having a plurality of nodes, where each portion of the invention may be located on a different node within the distributed system. In one embodiment of the invention, the node corresponds to a computer system. Alternatively, the node may correspond to a processor with associated physical memory. The node may alternatively correspond to a processor or micro-core of a processor with shared memory and/or resources. Further, computer readable program codes (e.g., software instructions) to perform embodiments of the invention may be stored on a computer readable medium. The computer readable medium may be a tangible computer readable medium, such as a compact disc (CD), a diskette, a tape, a flash memory device, random access memory (RAM), read only memory (ROM), or any other tangible medium.

Thus, one embodiment of the present invention is directed to system comprising: a non-transitory computer readable medium comprising computer readable program code stored thereon for causing a processor to determine the methylation status of the DNA; and compare the

methylation status to one or more methylated biomarkers selected from the following: cg04877910, cg09667289, cg05274650, cg11473616, cg16935734, cg27534624, cg21851713, cg15867829, cg15679829, cg08884979, cg09538401, cg26811868, cg05367028, cg19816080, cg20108357, cg25504868, cg11201447, cg19922137, cg14706317, cg15902830, cg10794973, cg10777887, cg03290131, cg07851269, cg11264947, cg00279406, cg23140965, cg03574652, cg03265671, cg24864241, cg01572891, cg00193963, cg14329285, cg17819990, cg17298239, cg23856138, cg21049501, cg11808936, cg25170591, cg17983632, cg08141142, cg19848599, cg25799109, cg07093324, cg16223546, cg07604732, cg12149606, cg08949329, cg27166177, cg26177041, cg09885851, cg22876153, cg21386992, cg02309772, cg02833180, cg20007890, cg04972244, cg02666955 and cg12102682. In a preferred embodiment, a report is generated based on the comparison providing guidance as to whether the sample is clear cell malignant, papillary malignant, chromophobe malignant, angiomylolipomas (AML) benign, or oncocytoma benign.

### Example 1

### Development of a DNA Methylation Classifier to Subtype Kidney Tumors

RCC and its subtypes (clear cell, papillary and chromophobe) account for about 90% of solid renal masses, with clear cell accounting for over 75%, while the remaining 10% are composed of other malignancies (sarcoma, lymphoma, carcinoid) and benign solid tumors (oncocytoma, angiomyolipoma) [19]. We built a classification model for kidney tumors using Illumina Infinium HumanMethylation450 (HM450) DNA methylation data from 697 tissues across six major subgroups: 283 clear cell, 81 papillary and 65 chromophobe RCC, 27 benign angiomylolipomas, 37 oncocytomas, and 204 normal kidney. DNA methylation data for the 429 malignant cancers and 204 adjacent normal kidney tissues were obtained from TCGA, and additional HM450 DNA methylation data were generated for 64 benign tumors from formalin-fixed paraffin embedded (FFPE) microdissected tumor samples collected at the University of Southern California. The average size of the benign tumors was 3.4 cm, with 72% qualifying as small renal mass (<4 cm).

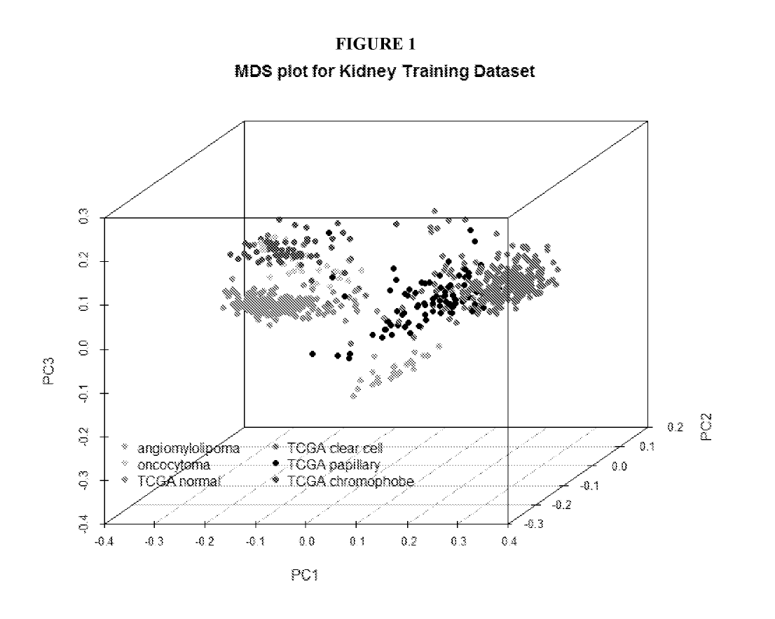

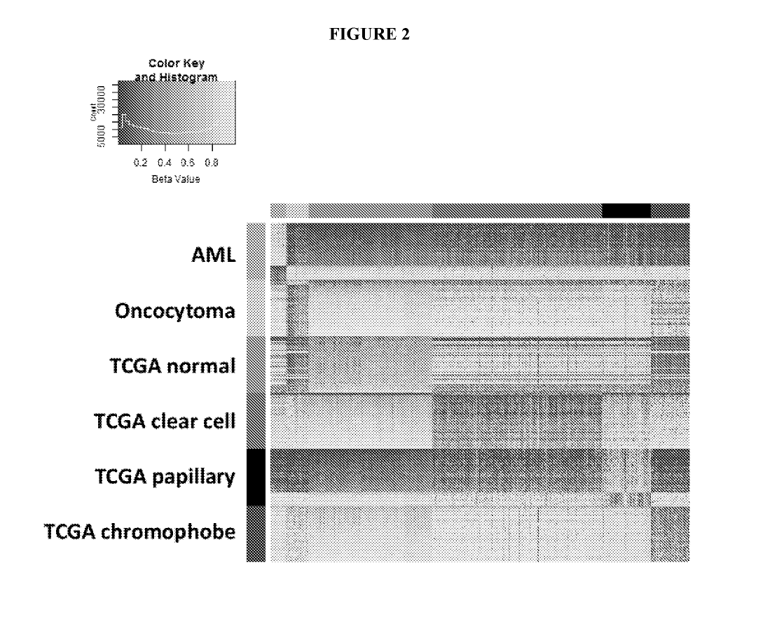

A multidimensional scaling plot of the 697 training samples shows clustering of normal kidney and well-defined tumor subtypes (FIG. 1). Angiomylolipomas (AML) form a distinct subgroup, oncocytomas and chromophobe RCCs cluster adjacent to one another, and clear cell and papillary RCCs cluster further away, indicative of unique DNA methylation profiles. For each subgroup, we selected the 100 CpG features with greatest separation of that subtype from all others, and combined all the lists. Interestingly, the six lists of features were unique and non-overlapping. FIG. 2 shows an ordered heatmap of the training samples for the 600 selected CpG features. Whereas the majority of loci predictive of normal kidney have intermediate DNA methylation levels, they were decreased in oncocytomas and chromophobe RCCs and increased in AML (benign) and clear cell and papillary RCCs. The majority of loci predictive for a single tumor subtype showed consistent increases or consistent decreases when compared to the other subtypes.

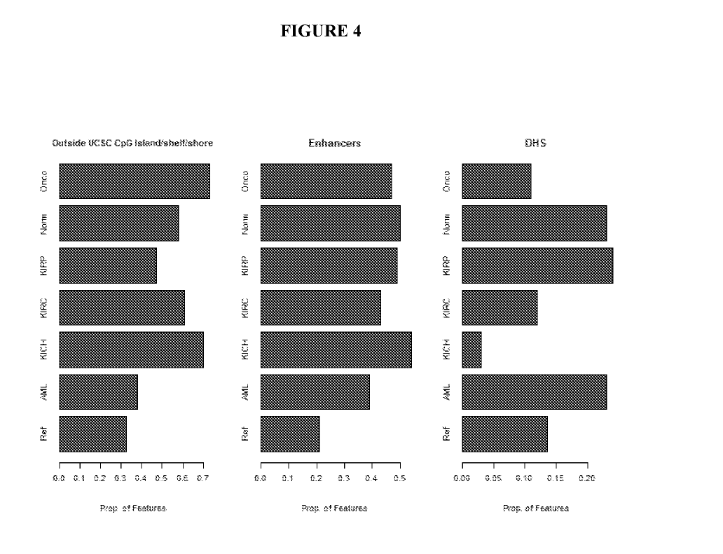

The selected features for all subgroups were enriched with features outside UCSC CpG islands, shelfs and shores, with greater than 2-fold enrichment for chromophobe RCCs and benign oncocytomas (70% and 73% vs 32% reference) (FIG. 4). Enhancers were enriched 1.9-fold in AML and more than 2-fold in malignant tumors, normal kidney and oncocytomas. DNaseI hypersensitive sites showed the greatest variation in enrichment, with chromophobe RCC showing 4.5-fold depletion while AML, papillary RCC and normal kidney showed a 1.7-fold enrichment. This finding suggests that alterations of DNA methylation in the tumor subtypes mainly happened in enhancers but not promoter regions.

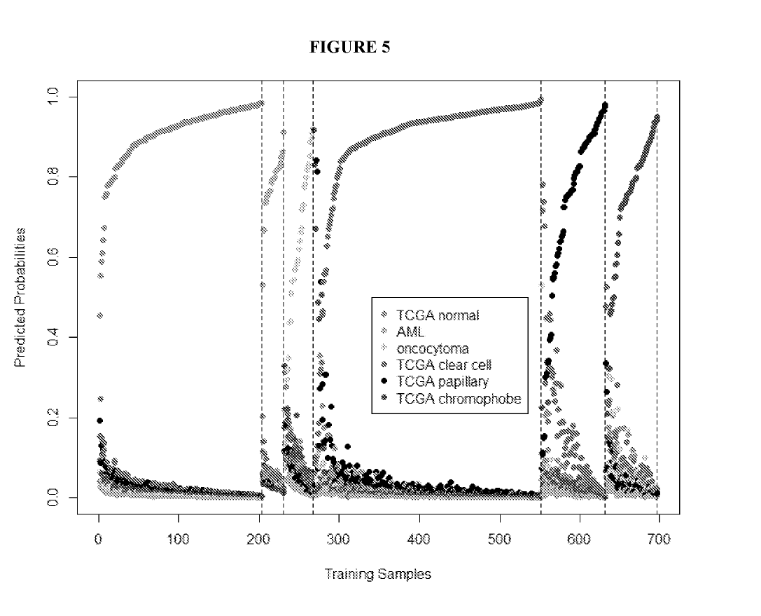

Furthermore, we built a multi-group classifier to predict tissue subtype, using an L1-penalty to reduce the DNA methylation feature set. The six groups were modeled using six equations, with each equation estimating the probability a sample belonged to one of the six groups and the sum of six probabilities equaling one. The final models used a combination of 59 variables: 2 for angiomylolipomas, 9 for oncocytomas, 11 for normal kidney, 13 for clear cell carcinomas, 14 for papillary and 10 for chromophobe RCC, with each model only selecting features from the subgroup-specific list. The classifier had 99.3% sensitivity and 99.6% specificity for the training data, detecting malignancy in 426 out of 429 cancers. Tumor subtype was predicted correctly in 95% of the training samples (407/429 malignant and 61/64 benign) (FIG. 5, Table 3).

### Example 2

### Using Ex Vivo SRM Needle Biopsies to Validate the Developed Classification Model

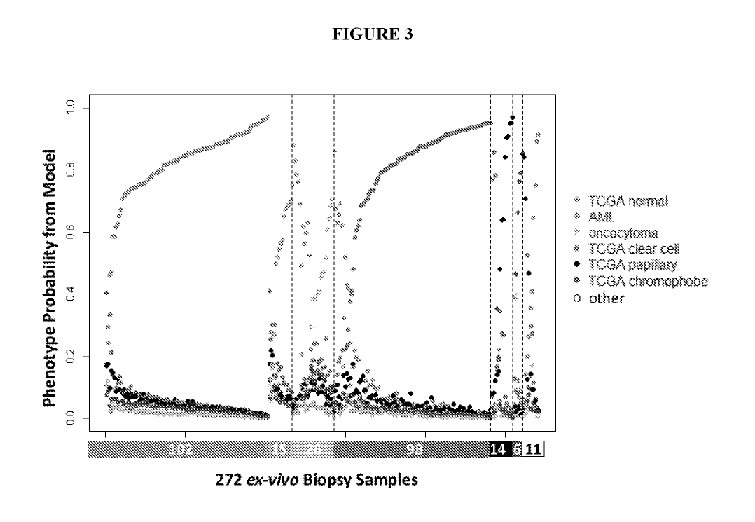

We obtained 272 ex vivo needle biopsy samples from 100 renal masses after nephrectomy (partial or total) at USC. Based on pathology reports, there were 70 malignant RMs and 30 benign RMs; in addition, 73 RMs were SRM (less than 4 cm) (Table 1). In general, three core biopsies were obtained from each patient: one from adjacent-normal tissue and two from the intact specimen using an 18-gauge side-cutting needle loaded on an automated biopsy gun. However, these numbers varied based on the availability of specimens across the patient set. For some ex vivo specimens, we only obtained one tumor needle biopsy. FIG. 3 shows the prediction probabilities for the six phenotypes using HM450 DNA methylation data from these 272 ex vivo needle biopsies. The probabilities were plotted for the six groups, the color bar at the bottom indicating the corresponding diagnosis from the pathologist. The maximum probability for each sample represents the predicted phenotype. Malignancy status was correctly predicted in 93% of samples, (86% of papillary, 91% of clear cell, 100% of chromophobe, 98% of normal kidney, 100% of oncocytoma, 80% of AML, and 64% of other benign tumors) (Table 2). Subtype was correctly estimated in 85% of samples (range: 58%-100%).

TABLE 1
Clinical and Pathological Characteristics of Samples
Included in the Analysis

| Variable | |
|---|---|
| N | 100 |
| Median age, years (Range) | 65 (21-87) |
| Gender (%) | |
| Male | 61.4% |
| Female | 38.6% |
| Median BMJ, kg/m² (Range) | 27.7 (16.9-47.1) |
| Median clinical tumor size, cm (Range) | 3 (1.3-10) |
| Mode of presentation (%) | |
| Incidental | 97% |
| Symptomatic | 3% |
| Surgical treatment (%) | |
| Partial Nephrectomy | 98% |
| Radical Nephrectomy | 2% |
| Median pathological tumor size, cm (Range) | 2.6 (1.0-9.5) |
| Final diagnosis (%) | |
| Benign lesion | 27% |
| Malignant lesion | 73% |
| pT Staging (%) | |
| pT1a | 70.8 |
| pT1b | 22.2 |
| pT2a | 2.8 |
| pT3a | 4.2 |
| pT3b | 0 |
| Lymph node involvement (%) | |
| pN0/Nx | 99% |
| pN+ | 1% |
| Distant metastasis (%) | |
| Absent | 100% |
| Present | 0% |

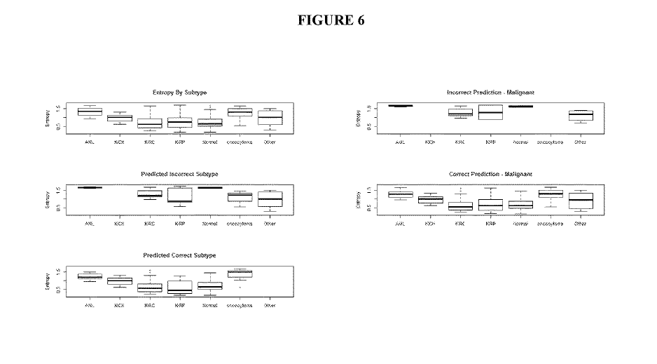

Classification error was evaluated as a function of the predicted probabilities. Entropy, the sum of p×log(p) for the six predictive probabilities p, captured classification uncertainty, with higher entropy for samples with more intermediate probability estimates and lower entropy for samples with greater discrimination in their probability estimates. Entropy varied by tumor subtype with benign AML and oncocytoma showing greater entropy compared to malignant tumors (FIG. 6). Not surprisingly, the entropy was also higher among samples predicted incorrectly than among those predicted correctly. Seventy-two percent of samples had a maximum probability above 0.70. Malignancy was correctly estimated in 98% and subtype in 96% of this high-confidence sample subset.

Out of the 100 tumors studied, 70 had DNA methylation data from two needle biopsies. The prediction based on multiple needle biopsies assigned an individual tumor to be malignant if the needle biopsy results for either measurement was malignant. Each sample was assigned the subtype from the needle biopsy with the highest probability estimate. In general, the results were highly reproducible with 62 of 70 tumors (89%) predicting identical subtypes from both biopsies. However, seven of the 62 concordant pairs (11%) were incorrectly predicted as normal kidney, of which two were missed malignant tumors (2 clear cell RCC), 3 ‘other’ benign, and 2 oncocytomas. Three malignant tumors with discordant needle biopsy results were correctly predicted as malignant when using two needle biopsies (2 clear cell, 1 papillary RCC). Overall, the sensitivity estimates at the tumor level reflected similar estimates at the sample level (Table 2). Sixty-four out of 70 (91%) tumors were correctly classified as malignant and 25 of 30 (83%) were correctly classified as benign.

TABLE 2
Validation of 271 ex vivo needle biopsies (100 patients).

| | Non-Malignant | | | | | Malignant | | |
|---|---|---|---|---|---|---|---|---|
| | Benign^{$} | | Oncocytoma | | Normal | Clear Cell | Papillary | Chromophobe |
| Based on Biopsy (N = 271) | | | | | | | | |
| Ex Vivo Biopsy (N) | 26 | | 26 | | 101 | 98 | 14 | 6 |
| Correctly Predicted | 11 | (73%)* | 15 | (58%) | 99 (98%) | 89 (91%) | 9 (64%) | 6 (100%) |
| Subtype (N, %) | | | | | | | | |
| Correctly Predicted Non- | 12 | (80%)* | 26 | (100%) | 99 (98%) | 89 (91%) | 12 (86%) | 6 (100%) |
| Malignant or Malignant | | | | | | | | |
| (N, %) | | | | | | | | |
| Based on Tumors (N = 100) | | | | | | | | |
| Tumors (N) | 14 | | 16 | | — | 59 | 8 | 3 |
| Correctly Predicted | 6 | (75%)* | 7 | (44%) | — | 53 (90%) | 5 (63%) | 3 (100%) |
| Subtype (N, %)¹ | | | | | | | | |
| Correctly Predicted Non- | 6 | (75%)* | 16 | (100%) | — | 54 (92%) | 7 (88%) | 3 (100%) |

Malignant or Malignant (N, %)

^{$}consists of angiomyolipoma and other uncommon non-malignant lesions (i.e. capillary hemangioma, renal tubular hyperplasia, etc.)
¹patient assigned subtype of biopsy with maximum posterior probability
*prediction only of angiomyolipoma (N = 15 ex vivo samples, N = 8 tumors)
)

TABLE 3
Training Data

| | | Correct | | | |
|---|---|---|---|---|---|
| | | Subtype | | Correct | |
| | Sample | Prediction | | Malignant/Non- | |
| | Size | (N) | (%) | Malignant (N) | (%) |
| Malignant | 429 | 407 | 94.9% | 426 | 99.3% |
| Clear Cell | 283 | 273 | 96.5% | 282 | 99.6% |
| Papillary | 81 | 72 | 88.9% | 80 | 98.8% |
| Chromophobe | 65 | 62 | 95.4% | 64 | 98.5% |
| Non-malignant | 268 | 265 | 99% | 267 | 99.6% |
| Normal | 204 | 204 | 100% | 204 | 100.0% |
| Benign | 64 | 61 | 95% | 63 | 98.4% |
| AML | 27 | 27 | 100% | 27 | 100.0% |
| oncocytoma | 37 | 34 | 92% | 36 | 97.3% |
| overall | 493 | 468 | 94.9% | | |

TABLE 4

| | SEQ | |
|---|---|---|
| | ID NO: | SEQUENCE |
| cg04877910 | 1 | CGCTCCAGCCACACCTAACTCAGGTTTCCCCAGGTAGGCGGGCATTCTTC |
| cg09667289 | 2 | CGCTTGCTGGACGCCGTTAGTGGTATTAACGGGAAGCCTCCAGACACTGA |
| cg02833180 | 3 | CGAGAGACCCCCAGCTGTGGAACTGAAGAACTGGTCTCCCACAAAGCTGA |
| cg02309772 | 4 | TTAGAGCCACACACATTTGTGAGAGCCAGCAGGGGCTGAGAACCGGTACG |
| cg22876153 | 5 | AACAGAGTGTGAGCCTGAAATACCCAAATACTTCAAATAAGACTTTCCCG |
| cg09885851 | 6 | CGGCAGGACTCGTGCTTCCCCTTAGATCACACAGATGTAAACCTGGGGAG |
| cg12102682 | 7 | CGGGGATTTCTGCTTATGATTCTAGTATGGTATACAGAGCCCAGTTTCCA |
| cg02666955 | 8 | CGGGATGTGTGGGTGAAGGGAACTAGCCACCTGTACTACCCCCTCACTTT |
| cg21386992 | 9 | AGGGGTCAGCAGAGCCCCCGTGGTCCAGACAGGCAGAGCCTCTGTGTCCG |
| cg20007890 | 10 | TGCCCACAGGCTGGCGGACGTCATGGCTCAGACCCACATAGGTGAGCACG |
| cg04972244 | 11 | CGTGGAATACCATTGTGTTTATTGATCAAGCCTGGCTTCGAGTGTGACAG |
| cg17983632 | 12 | CCGAGTTTGTGCAGGAGGTGCGTGGAACCCGGGTAGGCCAGGCCCCGTCG |
| cg12149606 | 13 | CGCGCCCGGCTAAGGCTGTTAATACCACTTTTTGTATCAGTAAGATCATG |
| cg25799109 | 14 | AATCAGATCTTTTGCCTTAGCAGATTCCCTTATTTAAGTTGTTGGAACCG |
| cg07093324 | 15 | CGCTAAGTCTAAGTAAGAGTCTGACTTCTCACTAGGAGCATGTCTGTTGT |
| cg27166177 | 16 | CTCAACCATGACGGTGACCAAGACCATAATCCCAGGTGGGAGGAGTCCCG |
| cg16223546 | 17 | CGCAAACACCGCCCTTGACTGTCTCTGCCTGTGGCTAGTGATGCAATTGT |
| cg19848599 | 18 | CGCGAGTTCCGTGGAGGTCATGCAAGCCCAGGCTAGGTCAGCATCAGGCT |
| cg26177041 | 19 | TTCATTTCCAGCCTTCTGCTTTCCTTTAAAGAGTCAGCTGTCATGTGCCG |
| cg08949329 | 20 | CCCAGTGGGCATGAACAAACTCTGGAGTGGATACAGCCTGCTGTACTTCG |
| cg08141142 | 21 | CGCGGCTAACTTATTCCGAGAATGCCGAGGAGTTGTCGTTTTTAGCTTTG |
| cg07604732 | 22 | CGGGTAGATCTGTTGCCTCAAAACTAGTGTACTGGTGCATATCCCAGAGC |
| cg10777887 | 23 | CGTGGAGGAGGGGAAATCCCATACCTCTTATTTAGCCCCAGAGCTCCAAC |
| cg11201447 | 24 | AGGATTGATACAACCCCCTTCTTGACTGATCAGAGCTTTAGAAAGATTCG |
| cg14706317 | 25 | CCACACTGTGGGCTCATGTCCCCTGTCCTGGAGGCAGCAACCGTGTGCCG |
| cg05367028 | 26 | ACCCCGAGACGGGTGCAGAATCAGCAGCGGGGATCATCCAGAGACTCTCG |
| cg19816080 | 27 | GGCACGTACCCGGTGATAAGGGCCACCCAGCAGGCAGGACGTGGGCTACG |
| cg11264947 | 28 | CGAGGCCTGGCACTGCGTCCTCAGAGCTTGTCTGTTGTTAGGTCCGTCGC |
| cg15902830 | 29 | CGCCAGCAACCACCACTGTTGGGGCAGCCCTGTGCCAGGCACTACAGGCC |
| cg03290131 | 30 | CGAGCCTGTGGCTTTCAAGCTGTGGACATCTGGCCTAGCTAGATTTCTAC |
| cg07851269 | 31 | TGCGGCATGCTCCTGAATCCGTCCTGGCTTCGAGCAGAACCAAGTGAGCG |
| cg20108357 | 32 | AGGGAATAGCTTACATTTTCATGGCGCCCCTTTTAAACAGGAAACCCACG |
| cg10794973 | 33 | GGAAGCTCACCTTCCACCCTGATGATCTACATACCCAATTGCCCTCTGCG |
| cg25504868 | 34 | CGGACTGGCCTTTGGAAGCTCCCTGCCCTGACGGGGTTGCCTGTCACCAC |
| cg19922137 | 35 | TGATGCGCTCGCCATGGACCGCACCAACTGGATGGCGGGGGCAGCAGACG |
| cg23856138 | 36 | CGGTTTAGGGAAGTTGTGGCCTTAGGAAAGACTTAAACAGCTGTTTTTGT |
| cg24864241 | 37 | CGGGGACTATTTACTCCTGATCCTAAGTGACAGCTTGGGGAGGGAGAGTC |
| cg21049501 | 38 | AAGGGACCCCAGAGGTGTCGGCGATGGGGGTGTACATGGGGCGCTGAGCG |
| cg03265671 | 39 | CGACCCTCAGAGTCCCACCCGGTGGCCTCCAAGCCCCGCTCCAGGATCCC |
| cg23140965 | 40 | AACACATAGACACTTGTTCTCTGCCTCTGGAATTACAAATATGTTACTCG |
| cg03574652 | 41 | CGGCTCTGCCGTCTGATGAATCTGTCCTTCCGAACCTCCAGAGGCTTCTC |
| cg17819990 | 42 | TGCTCTCCTGTTTGGGTTCATTGAGATGAACATCTTCCATGCTCTCCCCG |
| cg00193963 | 43 | CGGTTCTTAGTGACAAGGCAGTGAAGCCTCAGCTGGCTCCCTTGCACCTC |
| cg01572891 | 44 | GGTTCACCCGGAACAGAGGCTGAGGGCAGGGGGCAAGCAGCGTGGGGTCG |
| cg25170591 | 45 | CGCCTCCGACCCCCCTGCCTGGAAGCTGCTGTCCTTTGAGGGCTTCGGAG |
| cg14329285 | 46 | CGGTTGAGCCAAGCATTTCAGGGACAGCTGAGAAGAGCAGAAACTGAAGA |
| cg11808936 | 47 | CGGGGAGGTGGGAATCATTGGACCTGCATGCTGCCAGCTGTGAGATGCCA |
| cg17298239 | 48 | CGCCAGAACTCGGCCACCGAGAGCGCCGAGAGCATCGAGATCTACATCCC |
| cg00279406 | 49 | CGTGCAGGTGAACCAGAAAGTGGGCATGTTTGAGGCGCACATCCAGGCAC |
| cg08884979 | 50 | ATTCTCTGGTTTGGGAACATTAACCATTAACATTTCAAGAGGACCTTGCG |
| cg15867829 | 51 | CGTACCTTTCCAGCTAGTATCTGCAGCAGGTGGGAGAATGATAGTGATCT |
| cg11473616 | 52 | GCTGGTGTGGAGCTTCTGGCTCTAGGTGAGTGGCCTTTTTATAAACACCG |
| cg05274650 | 53 | AGGCCTGTTTCCTGACCCAGTTTTCTCCCCAATCTCTATTTAGCTGTTCG |
| cg27534624 | 54 | GACTGCAACCTGGGCCTCGTGATCAGCGACCCAGGGTGTGGCTGGTGGCG |
| cg21851713 | 55 | AGAAAGCTCAGGTGAGAGCAGGTCTTGCCTTGCTCTTAAAGTGCCAGACG |
| cg09538401 | 56 | TGAAGATCACAGTGAAGGAGCTGCTGCAGCAAAGACGGGCACACCAGGCG |
| cg15679829 | 57 | CGCCTGGAGAATCTGATTCAACACTGCTGGGTTGGGACCCAGGGTGCCTC |
| cg16935734 | 58 | CGCAAATGATTCAGCTGTGCATTTTGAGAGGAAAAATATATGTAAGGTTG |
| cg26811868 | 59 | CGGCCTAGTTGCACCAAGACTAGCAGCAATACTGACTACAGGTGTGCACC |

Taken together, the high specificity and sensitivity to predict not only benign and malignant but also the more detailed subtypes holds great promise for our DNA methylation classification model to develop into a DNA methylation-based assay for needle biopsy samples and potential liquid biopsy samples.

Treatment decision making for SRMs is an increasingly frequent and challenging clinical problem. The management of SRMs first requires accurate characterization, and then the options for treatment consist of active surveillance, surgical removal, or in situ ablation. This decision of the best treatment modality is based on clinical assessment of patient comorbidities and tumor characteristics. SRMs are represented by a heterogeneous group of benign and malignant histologic entities, with a range of biologic and clinical behaviors. However, the assessment of tumor malignancy generally relies on its size, shape, profile, as well as tissue enhancement on multiphasic computed tomography (CT) and magnetic resonance imaging (MRI). The use of renal tumor biopsies to obtain pathologic information to guide treatment decisions has been traditionally reserved for very selected cases of SRMs [20]. Before the advent of biologic-targeted therapies, there was also limited interest in the histologic characterization of advanced and metastatic renal tumors.

Needle biopsies have demonstrated an ability to improve kidney tissue selection while maintaining a low complication rate. However, a key limitation of needle biopsy is its high rate of false negative results. Combining molecular markers with histological results is one potential way to increase sensitivity. Our hypothesis is that by incorporating a DNA methylation assay derived from needle biopsies, patients will be placed into more appropriate treatment protocols. This could potentially reduce invasive and morbid SRM treatments, especially in the elderly or in patients with benign diseases. In fact, the American Urological Association recommendations for the management of localized renal tumors states the study of molecular and genetic profiling on percutaneous renal tumor biopsies as a research priority (see e.g., https://www.auanet.org/education/guidelines/renal-mass.cfm).

To identify candidate markers that are differentially methylated in RCC and build a classification model, we have taken advantage of the TCGA database [21-23], which contains Illumina Infinium HM450 DNA methylation data for 429 malignant RCCs and 204 normal-adjacent tissues. Although some of these tumors were too large to be classified as SRM (median clinical tumor size is 5.54 cm for clear cell renal carcinomas, 9.6 cm for chromophobe renal carcinoma, 5.35 cm for papillary renal carcinoma) [21-23], the large sample size allowed for the identification of predictive features and was instrumental in building a prediction model that we later validated using SRMs. However, size did not seem to be an issue since we successfully used this DNA methylation classification model to predict tumor types in ex vivo needle biopsies derived mainly from SRMs (73% of RMs). In addition, since non-malignant kidney tumors were not included in the TCGA, we included 64 non-malignant tumor samples from our laboratory to test whether there are specific patterns in the non-malignant tumors and their subtypes. These data strongly suggest that differential DNA methylation patterns exist not only between non-malignant and malignant tissues, but also among tumor subtypes. In particular, chromophobe RCC appears more similar to benign oncocytoma than the other malignant papillary and clear cell tumors, supporting our hypothesis that cancer-specific DNA methylation can be used as subtype-specific renal cancer biomarkers. In support of this, the six sets of probes used to predict each subtype are indeed non-overlapping, allowing for the identification of subtypes using DNA methylation data.

Normal kidney tissues were predicted with high specificity using DNA methylation data. Interestingly, the two normal kidney samples that were incorrectly classified as clear cell carcinomas came from patients with clear cell tumors, suggesting that the biopsy might have contained tumor cells from the patient. We also found the reverse, in which clear cell tumors were incorrectly classified as normal. However, these classification probabilities were greater than 20% for being clear cell, suggesting that the biopsy may not have captured a sufficient number of malignant cells. This suggests that the classifier accurately reflects cell mixtures based on the probabilities it assigns to the individual subgroups.

The highest error rates occurred for the benign tumor subtypes. The benign tumors most likely to be overcalled as malignant were those from subtypes that were too rare to be represented in our training dataset. The poor performance for AML and oncocytomas might be a result of the limited sample numbers (27 AML and 37 oncocytomas) for these subtypes and indicate a need to include more samples in future studies in order to establish a better separation pattern.

In summary, these data demonstrate that differential DNA methylation patterns exist not only between benign and malignant tissues, but also between tumor subtypes. These results fully support our hypothesis that cancer-specific DNA methylation can be used as subtype-specific RCC biomarkers. This DNA methylation classification model could allow for improved clinical management of RCC patients, in which unnecessary surgical procedures would be minimized for patients with benign lesions, thereby reducing patient-associated morbidity/mortality. Moreover, malignant lesions and their subtypes can be identified earlier, thus decreasing unnecessary radiation exposure from serial imaging and increasing the chance of preserving renal function.

### Example 3—Methods

### Patient Material, Samples, and Marking

In a prospectively-collected institutional review board (IRB)-approved database, ex vivo samples were collected from resected kidney tissue retrieved immediately post-operative. For each surgical specimen, three doublet biopsies were taken: two doublets in the mass, and one doublet in normal kidney parenchyma adjacent to the mass. One sample from each doublet was used for H&E preparation, and the other sample was used for DNA methylation analysis. FFPE-microdissected samples of 64 benign tumors were collected from our institution's IRB-approved renal tissue database. A trained pathologist reviewed each prospective kidney case and the block that contained the most pure pathology was selected for microdissection.

Training data include a total of 697 kidney samples consisting of 6 subtypes: 283 clear cell carcinomas, 81 papillary carcinomas, 65 chromophobe, 27 angiomylolipomas, 37 oncocytomas, and 204 normal kidney. HM450 profiles for the malignant cancers and normal kidney tissues were downloaded from the TCGA data portal (https://tcga-data.nci.nih.gov/tcga/), and supplemental HM450 DNA methylation profiles were generated for the FFPE-microdissected samples of 64 benign tumors collected at USC. A testing dataset comprised of 272 ex vivo needle biopsy samples collected from 100 patients after nephrectomy (partial or total) at USC. The 272 ex vivo samples included 98 clear cell, 14 papillary, 6 chromophobe, 101 normal kidney, 15 angiomylolipoma, 26 oncocytoma, 11 other benign. Seventy tumors had data from two needle biopsies.

### DNA Methylation Profiling

Genomic DNA (200-500 ng) from each FFPE sample was treated with sodium bisulfite and recovered using the Zymo EZ DNA methylation kit (Zymo Research) according to the manufacturer's specifications and eluted in 10 μl volume. An aliquot (1 μl) was removed for MethyLight-based quality control testing of bisulfite conversion completeness and the amount of bisulfite converted DNA available for the Illumina Infinium HM450 DNA methylation assay [24]. All samples passed the QC tests and were then repaired using the Illumina Restoration solution as described by the manufacturer. Each sample was then processed using the Infinium DNA methylation assay data production pipeline [25]. All HM450 profiles were generated at the USC Molecular Genomics Core Facility. All profiles were processed from DAT files using the minfi and wateRmelon packages in Bioconductor. We corrected for background intensity, dye bias and typeI/typeII design bias using ‘noob’ followed by BMIQ. Beta values from features with low signal intensity were assigned as missing and samples with more than 5% features missing were excluded. One sample was excluded from the test set for this reason. We applied the feature filter from TCGA omitting features due to SNPs, repetitive regions, or targeting CpH sites, also filtering features mapping to X or Y chromosomes. Features containing missing values in either training or testing dataset are excluded, leaving a final data set of 351,124 features.

### Pre-Selecting DNA Methylation Markers

We used the training data to select a priori a list of 100 features for each of the 6 renal tissue subtypes as a function of their differences in group means. Specifically, for each subtype, we ranked the features on the smallest difference in average Beta value between the given subtype and each remaining subtype. Then, the top 100 probes with the largest minimum absolute differences are selected. No feature was selected twice, resulting in a combined set of 600 features. These 600 features are displayed in a heatmap and used for training the classification model (FIG. 2).

### MDS Plot and Heatmap

A multidimensional scaling (MDS) plot of the 500 features with greatest median absolute deviation was created using the limma package. The heatmap shows a supervised clustering of the samples in the training data set for the 600 differentially-methylated CpG features. The columns represent samples and the rows represent predictive features, each ordered by group as follows: ex vivo angiomyolipoma, ex vivo oncocytoma, TCGA normal kidney, TCGA clear cell, TCGA papillary, and TCGA chromophobe RCCs.

### L₁-Penalized Classification Model

To predict tissue subtype we fit the L1-penalized multinomial logistic regression model using the GLMnet package in the R programming language. We provided as input the 600 features on 697 training samples, and performed 10-fold cross-validation to select the penalty parameter and reduced feature set. We tested the model on 272 ex vivo needle biopsy samples collected from 100 tumors after nephrectomy (partial or total) at USC.

The output of the GLMnet model is probabilities of belonging to each subgroup, as a function of the DNA methylation values of the selected features. For each sample, the probabilities for the six renal tissue subtypes sum to one and we assign each sample to the subgroup with the highest predicted probability. Classification error rates are evaluated using pathology as the gold standard. Error rates were assessed for two classifications: (1) discriminating malignant vs. non-malignant and (2) discriminating the six tissue subgroups. For the classification of malignant/non-malignant, clear cell, papillary, and chromophobic RCC are classified as malignant, and AML, oncocytoma and normal kidney as non-malignant.

The Cancer Genome Atlas data (KIRC, KICH, KIRP) are publicly available from the TCGA data portal (https://tcga-data.nci.nih.gov/tcga/). Additional data supporting the foregoing findings are available in the Open Science Framework repository, DOI 10.17605/OSF.IO/Y8BH2|ARK c7605/osf.io/y8bh2 at https://osf.io/y8bh2/.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

The following references are each relied upon and incorporated herein in their entirety.
1. Siegel R L, Miller K D, Jemal A: Cancer statistics, 2016. C A Cancer J Clin 2016, 66:7-30.
2. Jemal A, Siegel R, Ward E, Murray T, Xu J, Smigal C, Thun M J: Cancer statistics, 2006. C A Cancer J Clin 2006, 56:106-130.
3. Volpe A, Finelli A, Gill I S, Jewett M A, Martignoni G, Polascik T J, Remzi M, Uzzo R G: Rationale for percutaneous biopsy and histologic characterisation of renal tumours. Eur Urol 2012, 62:491-504.
4. Corcoran A T, Russo P, Lowrance W T, Asnis-Alibozek A, Libertino J A, Pryma D A, Divgi C R, Uzzo R G: A review of contemporary data on surgically resected renal masses—benign or malignant? Urology 2013, 81:707-713.
5. Cooperberg M R, Mallin K, Kane C J, Carroll P R: Treatment trends for stage I renal cell carcinoma. J Urol 2011, 186:394-399.
6. Frank I, Blute M L, Cheville J C, Lohse C M, Weaver A L, Zincke H: Solid renal tumors: an analysis of pathological features related to tumor size. J Urol 2003, 170:2217-2220.
7. Silverman S G, Mortele K J, Tuncali K, Jinzaki M, Cibas E S: Hyperattenuating renal masses: etiologies, pathogenesis, and imaging evaluation. Radiographics 2007, 27:1131-1143.
8. Kunkle D A, Crispen P L, Chen D Y, Greenberg R E, Uzzo R G: Enhancing renal masses with zero net growth during active surveillance. J Urol 2007, 177:849-853; discussion 853-844.
9. Kelley C M, Cohen M B, Raab S S: Utility of fine-needle aspiration biopsy in solid renal masses. Diagn Cytopathol 1996, 14:14-19.
10. Barocas D A, Rohan S M, Kao J, Gurevich R D, Del Pizzo J J, Vaughan E D, Jr., Akhtar M, Chen Y T, Scherr D S: Diagnosis of renal tumors on needle biopsy specimens by histological and molecular analysis. J Urol 2006, 176:1957-1962.
11. Phe V, Yates D R, Renard-Penna R, Cussenot O, Roupret M: Is there a contemporary role for percutaneous needle biopsy in the era of small renal masses? BJU Int 2012, 109:867-872.
12. Jones P A, Baylin S B: The epigenomics of cancer. Cell 2007, 128:683-692.
13. deVos T, Tetzner R, Model F, Weiss G, Schuster M, Distler J, Steiger K V, Grutzmann R, Pilarsky C, Habermann J K, et al: Circulating methylated SEPT9 DNA in plasma is a biomarker for colorectal cancer. Clin Chem 2009, 55:1337-1346.
14. Payne S R, Serth J, Schostak M, Kamradt J, Strauss A, Thelen P, Model F, Day J K, Liebenberg V, Morotti A, et al: DNA methylation biomarkers of prostate cancer: confirmation of candidates and evidence urine is the most sensitive body fluid for non-invasive detection. Prostate 2009, 69:1257-1269.
15. Khakpour G, Pooladi A, Izadi P, Noruzinia M, Tavakkoly Bazzaz J: DNA methylation as a promising landscape: A simple blood test for breast cancer prediction. Tumour Biol 2015, 36:4905-4912.
16. Su S F, de Castro Abreu A L, Chihara Y, Tsai Y, Andreu-Vieyra C, Daneshmand S, Skinner E C, Jones P A, Siegmund K D, Liang G: A panel of three markers hyper- and hypomethylated in urine sediments accurately predicts bladder cancer recurrence. Clin Cancer Res 2014, 20:1978-1989.
17. Morris M R, Maher E R: Epigenetics of renal cell carcinoma: the path towards new diagnostics and therapeutics. Genome Med 2010, 2:59.
18. Morris M R, Ricketts C J, Gentle D, McRonald F, Carli N, Khalili H, Brown M, Kishida T, Yao M, Banks R E, et al: Genome-wide methylation analysis identifies epigenetically inactivated candidate tumour suppressor genes in renal cell carcinoma. Oncogene 2011, 30:1390-1401.
19. Murai M, Oya M: Renal cell carcinoma: etiology, incidence and epidemiology. Curr Opin Urol 2004, 14:229-233.
20. Herts B R, Baker M E: The current role of percutaneous biopsy in the evaluation of renal masses. Semin Urol Oncol 1995, 13:254-261.
21. Cancer Genome Atlas Research N: Comprehensive molecular characterization of clear cell renal cell carcinoma. Nature 2013, 499:43-49.
22. Davis C F, Ricketts C J, Wang M, Yang L, Cherniack A D, Shen H, Buhay C, Kang H, Kim S C, Fahey C C, et al: The somatic genomic landscape of chromophobe renal cell carcinoma. Cancer Cell 2014, 26:319-330.
23. Cancer Genome Atlas Research N, Linehan W M, Spellman P T, Ricketts C J, Creighton C J, Fei S S, Davis C, Wheeler D A, Murray B A, Schmidt L, et al: Comprehensive Molecular Characterization of Papillary Renal-Cell Carcinoma. N Engl J Med 2016, 374:135-145.
24. Campan M, Weisenberger D J, Trinh B, Laird P W: MethyLight. Methods Mol Biol 2009, 507:325-337.
25. Bibikova M, Barnes B, Tsan C, Ho V, Klotzle B, Le J M, Delano D, Zhang L, Schroth G P, Gunderson K L, et al: High density DNA methylation array with single CpG site resolution. Genomics 2011, 98:288-295.
26. Chopra S, Liu J, Alemozaffar M, Nichols P, Aron M, Weisenberger D, Collings C, Syan S, Hu B, Desai M M, Aron M, Duddalwar V, Gill I S, Liang G, Siegmund K. Improving needle biopsy accuracy in small renal mass using tumor-specific DNA methylation markers. Oncotarget 2016; doi: 10.18632/oncotarget.12276.

What is claimed is:

1. A method of classifying kidney tumors comprising:
obtaining a sample from a subject;
isolating DNA from the sample;
determining the methylation status of the DNA; and comparing the methylation status of the DNA to one or more methylated biomarkers selected from the group consisting of cg04877910, cg09667289, cg05274650, cg11473616, cg16935734, cg27534624, cg21851713, cg15867829, cg15679829, cg08884979, cg09538401, cg26811868, cg05367028, cg19816080, cg20108357, cg25504868, cg11201447, cg19922137, cg14706317, cg15902830, cg10794973, cg10777887, cg03290131, cg07851269, cg11264947, cg00279406, cg23140965, cg03574652, cg03265671, cg24864241, cg01572891, cg00193963, cg14329285, cg17819990, cg17298239, cg23856138, cg21049501, cg11808936, cg25170591, cg17983632, cg08141142, cg19848599, cg25799109, cg07093324, cg16223546, cg07604732, cg12149606, cg08949329, cg27166177, cg26177041, cg09885851, cg22876153, cg21386992, cg02309772, cg02833180, cg20007890, cg04972244, cg02666955 and cg12102682,
wherein the methylated biomarker comprises a sequence region that extends up to 250 base pairs upstream and downstream from the methylated biomarker, and
wherein the comparison indicates whether the sample is clear cell malignant, papillary malignant, chromophobe malignant, angiomylolipomas (AML) benign, or oncocytoma benign.

2. The method of claim 1, wherein the sample is a biopsy sample.

3. The method of claim 2, wherein the biopsy is from a small renal mass (SRM).

4. The method of claim 1, wherein two or more methylated biomarkers are selected.

5. The method of claim 1, wherein the sample is selected from the group consisting of blood, plasma and urine.

6. The method of claim 1, wherein the sequence region extends up to 100 base pairs upstream and downstream from the methylated biomarker.

7. The method of claim 1, wherein the sequence region extends 0 base pairs upstream and downstream from the methylated biomarker.

8. The method of claim 1, wherein five or more methylated biomarkers are selected.

9. The method of claim 1, wherein fifteen or more methylated biomarkers are selected.

10. A method of identifying subjects having renal cancer comprising:
obtaining a sample from a subject;
isolating DNA from the sample;
determining the methylation status of the DNA; and comparing the methylation status of the DNA to one or more methylated biomarkers selected from the group consisting of cg04877910, cg09667289, cg05274650, cg11473616, cg16935734, cg27534624, cg21851713, cg15867829, cg15679829, cg08884979, cg09538401, cg26811868, cg05367028, cg19816080, cg20108357, cg25504868, cg11201447, cg19922137, cg14706317, cg15902830, cg10794973, cg10777887, cg03290131, cg07851269, cg11264947, cg00279406, cg23140965, cg03574652, cg03265671, cg24864241, cg01572891, cg00193963, cg14329285, cg17819990, cg17298239, cg23856138, cg21049501, cg11808936, cg25170591, cg17983632, cg08141142, cg19848599, cg25799109, cg07093324, cg16223546, cg07604732, cg12149606, cg08949329, cg27166177, cg26177041, cg09885851, cg22876153, cg21386992, cg02309772, cg02833180, cg20007890, cg04972244, cg02666955 and cg12102682,
wherein the methylated biomarker comprises a sequence region that extends up to 250 base pairs upstream and downstream from the methylated biomarker, and
wherein the comparison indicates whether the sample is normal or malignant.

11. The method of claim 10, wherein the sample is a biopsy sample.

12. The method of claim 11, wherein the biopsy is from a small renal mass (SRM).

13. The method of claim 10, wherein two or more methylated biomarkers are selected.

14. The method of claim 10, wherein the sample is selected from the group consisting of blood, plasma and urine.

15. The method of claim 10, wherein the sequence region extends up to 100 base pairs upstream and downstream from the methylated biomarker.

16. The method of claim 10, wherein the sequence region extends 0 base pairs upstream and downstream from the methylated biomarker.

17. The method of claim 10, wherein five or more methylated biomarkers are selected.

18. The method of claim 10, wherein fifteen or more methylated biomarkers are selected.

19. A composition comprising one or more methylated biomarkers selected from the group consisting of cg04877910, cg09667289, cg05274650, cg11473616, cg16935734, cg27534624, cg21851713, cg15867829, cg15679829, cg08884979, cg09538401, cg26811868, cg05367028, cg19816080, cg20108357, cg25504868, cg11201447, cg19922137, cg14706317, cg15902830, cg10794973, cg10777887, cg03290131, cg07851269, cg11264947, cg00279406, cg23140965, cg03574652, cg03265671, cg24864241, cg01572891, cg00193963, cg14329285, cg17819990, cg17298239, cg23856138, cg21049501, cg11808936, cg25170591, cg17983632, cg08141142, cg19848599, cg25799109, cg07093324, cg16223546, cg07604732, cg12149606, cg08949329, cg27166177, cg26177041, cg09885851, cg22876153, cg21386992, cg02309772, cg02833180, cg20007890, cg04972244, cg02666955 and cg12102682.

20. The composition of claim 19, wherein the composition is used in an assay to determine whether a sample is clear cell malignant, papillary malignant, chromophobe malignant, angiomylolipomas (AML) benign, or oncocytoma benign.

21. The composition of claim 19, wherein the composition is used in an assay to determine whether a sample is normal or malignant.


SEQUENCE LISTING (59 sequences)

SEQ ID NO: 1 - DNA, 50 bp, Artificial Sequence
  Synthetic oligonucleotide
cgctccagccacacctaactcaggtttccccaggtaggcgggcattcttc

SEQ ID NO: 2 - DNA, 50 bp, Artificial Sequence
  Synthetic oligonucleotide
cgcttgctggacgccgttagtggtattaacgggaagcctccagacactga

SEQ ID NO: 3 - DNA, 50 bp, Artificial Sequence
  Synthetic oligonucleotide
cgagagacccccagctgtggaactgaagaactggtctcccacaaagctga

SEQ ID NO: 4 - DNA, 50 bp, Artificial Sequence
  Synthetic oligonucleotide
ttagagccacacacatttgtgagagccagcaggggctgagaaccggtacg

SEQ ID NO: 5 - DNA, 50 bp, Artificial Sequence
  Synthetic oligonucleotide
aacagagtgtgagcctgaaatacccaaatacttcaaataagactttcccg

SEQ ID NO: 6 - DNA, 50 bp, Artificial Sequence
  Synthetic oligonucleotide
cggcaggactcgtgcttccccttagatcacacagatgtaaacctggggag

SEQ ID NO: 7 - DNA, 50 bp, Artificial Sequence
  Synthetic oligonucleotide
cggggatttctgcttatgattctagtatggtatacagagcccagtttcca

SEQ ID NO: 8 - DNA, 50 bp, Artificial Sequence
  Synthetic oligonucleotide
cgggatgtgtgggtgaagggaactagccacctgtactaccccctcacttt

SEQ ID NO: 9 - DNA, 50 bp, Artificial Sequence
  Synthetic oligonucleotide
aggggtcagcagagcccccgtggtccagacaggcagagcctctgtgtccg

SEQ ID NO: 10 - DNA, 50 bp, Artificial Sequence
  Synthetic oligonucleotide
tgcccacaggctggcggacgtcatggctcagacccacataggtgagcacg

SEQ ID NO: 11 - DNA, 50 bp, Artificial Sequence
  Synthetic oligonucleotide
cgtggaataccattgtgtttattgatcaagcctggcttcgagtgtgacag

SEQ ID NO: 12 - DNA, 50 bp, Artificial Sequence
  Synthetic oligonucleotide
ccgagtttgtgcaggaggtgcgtggaacccgggtaggccaggccccgtcg

SEQ ID NO: 13 - DNA, 50 bp, Artificial Sequence
  Synthetic oligonucleotide
cgcgcccggctaaggctgttaataccactttttgtatcagtaagatcatg

SEQ ID NO: 14 - DNA, 50 bp, Artificial Sequence
  Synthetic oligonucleotide
aatcagatcttttgccttagcagattcccttatttaagttgttggaaccg

SEQ ID NO: 15 - DNA, 50 bp, Artificial Sequence
  Synthetic oligonucleotide
cgctaagtctaagtaagagtctgacttctcactaggagcatgtctgttgt

SEQ ID NO: 16 - DNA, 50 bp, Artificial Sequence
  Synthetic oligonucleotide
ctcaaccatgacggtgaccaagaccataatcccaggtgggaggagtcccg

SEQ ID NO: 17 - DNA, 50 bp, Artificial Sequence
  Synthetic oligonucleotide
cgcaaacaccgcccttgactgtctctgcctgtggctagtgatgcaattgt

SEQ ID NO: 18 - DNA, 50 bp, Artificial Sequence
  Synthetic oligonucleotide
cgcgagttccgtggaggtcatgcaagcccaggctaggtcagcatcaggct

SEQ ID NO: 19 - DNA, 50 bp, Artificial Sequence
  Synthetic oligonucleotide
ttcatttccagccttctgctttcctttaaagagtcagctgtcatgtgccg

SEQ ID NO: 20 - DNA, 50 bp, Artificial Sequence
  Synthetic oligonucleotide
cccagtgggcatgaacaaactctggagtggatacagcctgctgtacttcg

SEQ ID NO: 21 - DNA, 50 bp, Artificial Sequence
  Synthetic oligonucleotide
cgcggctaacttattccgagaatgccgaggagttgtcgtttttagctttg

SEQ ID NO: 22 - DNA, 50 bp, Artificial Sequence
  Synthetic oligonucleotide
cgggtagatctgttgcctcaaaactagtgtactggtgcatatcccagagc

SEQ ID NO: 23 - DNA, 50 bp, Artificial Sequence
  Synthetic oligonucleotide
cgtggaggaggggaaatcccatacctcttatttagccccagagctccaac

SEQ ID NO: 24 - DNA, 50 bp, Artificial Sequence
  Synthetic oligonucleotide
aggattgatacaacccccttcttgactgatcagagctttagaaagattcg

SEQ ID NO: 25 - DNA, 50 bp, Artificial Sequence
  Synthetic oligonucleotide
ccacactgtgggctcatgtcccctgtcctggaggcagcaaccgtgtgccg

SEQ ID NO: 26 - DNA, 50 bp, Artificial Sequence
  Synthetic oligonucleotide
accccgagacgggtgcagaatcagcagcggggatcatccagagactctcg

SEQ ID NO: 27 - DNA, 50 bp, Artificial Sequence
  Synthetic oligonucleotide
ggcacgtacccggtgataagggccacccagcaggcaggacgtgggctacg

SEQ ID NO: 28 - DNA, 50 bp, Artificial Sequence
  Synthetic oligonucleotide
cgaggcctggcactgcgtcctcagagcttgtctgttgttaggtccgtcgc

SEQ ID NO: 29 - DNA, 50 bp, Artificial Sequence
  Synthetic oligonucleotide
cgccagcaaccaccactgttggggcagccctgtgccaggcactacaggcc

SEQ ID NO: 30 - DNA, 50 bp, Artificial Sequence
  Synthetic oligonucleotide
cgagcctgtggctttcaagctgtggacatctggcctagctagatttctac

SEQ ID NO: 31 - DNA, 50 bp, Artificial Sequence
  Synthetic oligonucleotide
tgcggcatgctcctgaatccgtcctggcttcgagcagaaccaagtgagcg

SEQ ID NO: 32 - DNA, 50 bp, Artificial Sequence
  Synthetic oligonucleotide
agggaatagcttacattttcatggcgccccttttaaacaggaaacccacg

SEQ ID NO: 33 - DNA, 50 bp, Artificial Sequence
  Synthetic oligonucleotide
ggaagctcaccttccaccctgatgatctacatacccaattgccctctgcg

SEQ ID NO: 34 - DNA, 50 bp, Artificial Sequence
  Synthetic oligonucleotide
cggactggcctttggaagctccctgccctgacggggttgcctgtcaccac

SEQ ID NO: 35 - DNA, 50 bp, Artificial Sequence
  Synthetic oligonucleotide
tgatgcgctcgccatggaccgcaccaactggatggcgggggcagcagacg

SEQ ID NO: 36 - DNA, 50 bp, Artificial Sequence
  Synthetic oligonucleotide
cggtttagggaagttgtggccttaggaaagacttaaacagctgtttttgt

SEQ ID NO: 37 - DNA, 50 bp, Artificial Sequence
  Synthetic oligonucleotide
cggggactatttactcctgatcctaagtgacagcttggggagggagagtc

SEQ ID NO: 38 - DNA, 50 bp, Artificial Sequence
  Synthetic oligonucleotide
aagggaccccagaggtgtcggcgatgggggtgtacatggggcgctgagcg

SEQ ID NO: 39 - DNA, 50 bp, Artificial Sequence
  Synthetic oligonucleotide
cgaccctcagagtcccacccggtggcctccaagccccgctccaggatccc

SEQ ID NO: 40 - DNA, 50 bp, Artificial Sequence
  Synthetic oligonucleotide
aacacatagacacttgttctctgcctctggaattacaaatatgttactcg

SEQ ID NO: 41 - DNA, 50 bp, Artificial Sequence
  Synthetic oligonucleotide
cggctctgccgtctgatgaatctgtccttccgaacctccagaggcttctc

SEQ ID NO: 42 - DNA, 50 bp, Artificial Sequence
  Synthetic oligonucleotide
tgctctcctgtttgggttcattgagatgaacatcttccatgctctccccg

SEQ ID NO: 43 - DNA, 50 bp, Artificial Sequence
  Synthetic oligonucleotide
cggttcttagtgacaaggcagtgaagcctcagctggctcccttgcacctc

SEQ ID NO: 44 - DNA, 50 bp, Artificial Sequence
  Synthetic oligonucleotide
ggttcacccggaacagaggctgagggcagggggcaagcagcgtggggtcg

SEQ ID NO: 45 - DNA, 50 bp, Artificial Sequence
  Synthetic oligonucleotide
cgcctccgacccccctgcctggaagctgctgtcctttgagggcttcggag

SEQ ID NO: 46 - DNA, 50 bp, Artificial Sequence
  Synthetic oligonucleotide
cggttgagccaagcatttcagggacagctgagaagagcagaaactgaaga

SEQ ID NO: 47 - DNA, 50 bp, Artificial Sequence
  Synthetic oligonucleotide
cggggaggtgggaatcattggacctgcatgctgccagctgtgagatgcca

SEQ ID NO: 48 - DNA, 50 bp, Artificial Sequence
  Synthetic oligonucleotide
cgccagaactcggccaccgagagcgccgagagcatcgagatctacatccc

SEQ ID NO: 49 - DNA, 50 bp, Artificial Sequence
  Synthetic oligonucleotide
cgtgcaggtgaaccagaaagtgggcatgtttgaggcgcacatccaggcac

SEQ ID NO: 50 - DNA, 50 bp, Artificial Sequence
  Synthetic oligonucleotide
attctctggtttgggaacattaaccattaacatttcaagaggaccttgcg

SEQ ID NO: 51 - DNA, 50 bp, Artificial Sequence
  Synthetic oligonucleotide
cgtacctttccagctagtatctgcagcaggtgggagaatgatagtgatct

SEQ ID NO: 52 - DNA, 50 bp, Artificial Sequence
  Synthetic oligonucleotide
gctggtgtggagcttctggctctaggtgagtggcctttttataaacaccg

SEQ ID NO: 53 - DNA, 50 bp, Artificial Sequence
  Synthetic oligonucleotide
aggcctgtttcctgacccagttttctccccaatctctatttagctgttcg

SEQ ID NO: 54 - DNA, 50 bp, Artificial Sequence
  Synthetic oligonucleotide
gactgcaacctgggcctcgtgatcagcgacccagggtgtggctggtggcg

SEQ ID NO: 55 - DNA, 50 bp, Artificial Sequence
  Synthetic oligonucleotide
agaaagctcaggtgagagcaggtcttgccttgctcttaaagtgccagacg

SEQ ID NO: 56 - DNA, 50 bp, Artificial Sequence
  Synthetic oligonucleotide
tgaagatcacagtgaaggagctgctgcagcaaagacgggcacaccaggcg

SEQ ID NO: 57 - DNA, 50 bp, Artificial Sequence
  Synthetic oligonucleotide
cgcctggagaatctgattcaacactgctgggttgggacccagggtgcctc

SEQ ID NO: 58 - DNA, 50 bp, Artificial Sequence
  Synthetic oligonucleotide
cgcaaatgattcagctgtgcattttgagaggaaaaatatatgtaaggttg

SEQ ID NO: 59 - DNA, 50 bp, Artificial Sequence
  Synthetic oligonucleotide
cggcctagttgcaccaagactagcagcaatactgactacaggtgtgcacc